(19) United States
(12) Patent Application Publication
(10) Pub. No.: US 2021/0357860 A1
(43) Pub. Date: Nov. 18, 2021

(54) EXPERT SELECTION SYSTEM AND EXPERT SELECTION PROGRAM

(71) Applicant: KONICA MINOLTA, INC., Tokyo (JP)

(72) Inventor: Kazuki TATSUMI, Tokyo (JP)

(73) Assignee: KONICA MINOLTA, INC., Tokyo (JP)

(21) Appl. No.: 17/232,294

(22) Filed: Apr. 16, 2021

(30) Foreign Application Priority Data
May 13, 2020 (JP) 2020-084422

(51) Int. Cl. G06Q 10/10; G06F 16/28

(52) U.S. Cl. CPC G06Q 10/103; G06Q 10/105; G16H 70/20; G06F 16/288; G06Q 10/109

(57) ABSTRACT

There are provided an expert selection system and an expert selection program which are capable of selecting an expert for a proposal document. The expert selection system has a proposal information acquisition part, a storage part, and an expert selection part. The proposal information acquisition part acquires proposal information in which relationships between elements included in proposal data of a proposer are structured. The storage part stores knowledge information in which relationships between elements corresponding to a plurality of pieces of information forming knowledge are structured for each member. The expert selection part compares the proposal information and the knowledge information associated with members other than the proposer of the proposal data for each member and selects an expert who has knowledge regarding the proposal data from a plurality of the members on the basis of a comparison result.

### CROSS-REFERENCE TO RELATED APPLICATION

The entire disclosure of Japanese Patent Application No. 2020-084422, filed on May 13, 2020, is incorporated herein by reference in its entirety.

### BACKGROUND

### 1. Technological Field

The present invention relates to an expert selection system and an expert selection program.

### 2. Description of the Related Art

Normally, in an organization such as a government office or an enterprise, an approver who has an approval authority for a document created in a business performs an approval procedure. In the approval procedure, it is common that a superior in the hierarchy in one group (hereinafter referred to as “cluster”) such as a department or a team plays the role of the approver. In that case, the approval procedure is based on the premise that a superior (leader) of the cluster has knowledge about the business contents of a subordinate (general member). Further, the approval procedure may be performed across clusters.

In relation to this, JP 2015-170263 A discloses a workflow integration system of which the purpose is to make, when integrating workflow systems of a plurality of enterprises, setting of approval authority and authentication processing for approvers efficient by absorbing a difference in job between companies when integrating the workflow systems of multiple companies.

JP 2015-95128 A discloses a technique for classifying approvers into levels and determining an approver on the basis of the levels of the approvers and the status of entry and exit when executing jobs that require approval.

### SUMMARY

However, in a case where diverse human resources are gathered in an organization due to the complexity of business contents, members in the same cluster do not necessarily perform the same business or business closely related to each other. For example, this is applied to a case where human resources with specialties in different fields are gathered in one department, a case where members who speak different languages are enrolled, or the like. In such a case, a superior of the organization in a certain cluster may not have knowledge of the business contents of a subordinate.

For example, in a case where a superior who has previously played the role of an approver transfers the role of approval to a successor due to, for example, transfer or retirement, the successor may not have knowledge about the business contents of a subordinate.

As described above, although the superior of a certain cluster does not have knowledge regarding the document (proposal document) created by a member (proposer) of the cluster, the superior may be the approver of the proposal document only because of a level or position in the cluster. However, in that case, there is a risk that the superior cannot fully understand the contents of the proposal and cannot sufficiently fulfill the role of the approver.

The present invention has been made in view of the above circumstances, and an object of the present invention is to provide an expert selection system and an expert selection program which are capable of selecting an expert who has knowledge about a proposal document.

To achieve at least one of the above-mentioned objects, according to an aspect of the present invention, an expert selection system reflecting one aspect of the present invention comprises: a storage part that stores knowledge information in which relationships between elements corresponding to a plurality of pieces of information forming knowledge are structured for each member; and a hardware processor. The hardware processor acquires proposal information in which relationships between elements included in proposal data of a proposer are structured, compares the proposal information and the knowledge information associated with members other than the proposer of the proposal data for each member, and selects an expert who has knowledge regarding the proposal data from a plurality of the members on the basis of a comparison result.

A non-transitory recording medium reflecting one aspect of the present invention stores a computer readable program causing a computer to perform: acquiring proposal information in which relationships between elements included in proposal data of a proposer are structured; acquiring knowledge information in which relationships between elements corresponding to a plurality of pieces of information forming knowledge are structured; and comparing the proposal information of the proposer and the knowledge information associated with members other than the proposer for each of the members and selecting an expert who has knowledge regarding the proposal data from a plurality of the members on the basis of a comparison result.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a schematic block diagram illustrating a hardware configuration of an expert selection system according to one embodiment;

FIG. 2 is a schematic block diagram illustrating a hardware configuration of the expert selection device illustrated in FIG. 1;

FIG. 3 is a schematic block diagram illustrating main functional parts of the expert selection device illustrated in FIG. 2;

FIG. 4 is a network diagram illustrating a part of knowledge information stored in a knowledge database illustrated in FIG. 1;

FIG. 5 is a schematic block diagram illustrating a hardware configuration of a client terminal illustrated in FIG. 1;

FIG. 6 is a flowchart illustrating a processing procedure of a control method of the expert selection system of one embodiment;

FIG. 7 is a schematic diagram illustrating a report created by a proposer;

FIG. 8 is a network diagram illustrating proposal information corresponding to the report of FIG. 7;

FIG. 9 is a schematic diagram illustrating display of information regarding experts;

FIG. 10 is a subroutine flowchart illustrating a process of selecting an expert;

FIG. 11 is a conceptual diagram illustrating a similarity between the proposal information and the knowledge information;

FIG. 12 is a network diagram illustrating a comparison between the proposal information and the knowledge information; and

FIG. 13 is a conceptual diagram illustrating an inclusion relationship between the proposal information and the knowledge information.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, an embodiment of the present invention will be described with reference to the accompanying drawings. Incidentally, in the description of the drawings, the same elements are denoted by the same reference numerals, and redundant description is omitted. Further, the dimensional ratios in the drawings may are exaggerated for convenience of description and be different from the actual ratios.

<Expert Selection System 100>

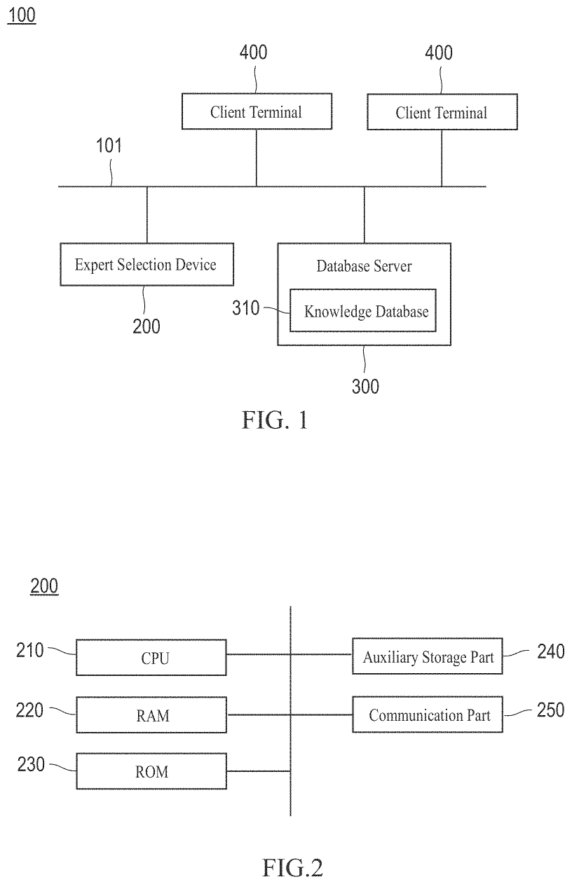

FIG. 1 is a schematic block diagram illustrating a hardware configuration of an expert selection system 100 according to one embodiment, and FIG. 2 is a schematic block diagram illustrating a hardware configuration of an expert selection device 200 illustrated in FIG. 1.

As illustrated in FIG. 1, the expert selection system 100 has the expert selection device 200, a database server 300, and a plurality of client terminals 400 which are connected to be able to communicate with each other via a communication network 101 including, for example, LAN (Local Area Network), WAN (Wide Area Network), the Internet, and the like. In this embodiment, it is assumed that the proposer of the proposal document and the direct supervisor (hereinafter, also simply referred to as “supervisor”) of the proposer use one client terminal 400 each. Incidentally, the client terminal 400 may not be included in the expert selection system 100.

<Expert Selection Device 200>

The expert selection device 200 functions as a server (computer). As illustrated in FIG. 2, the expert selection device 200 includes a CPU (Central Processing Unit) 210, a RAM (Random Access Memory) 220, a ROM (Read Only Memory) 230, an auxiliary storage part 240, a communication part 250, and the like.

The CPU 210 executes a program such as an OS (Operating System) deployed in the RAM 220 and an expert selection program, and controls the operation of the expert selection device 200. The expert selection program is stored in the ROM 230 or the auxiliary storage part 240 in advance. Further, the RAM 220 stores data and the like temporarily generated by the processing of the CPU 210. The ROM 230 stores the program executed by the CPU 210, the data used for executing the program, parameters, and the like.

The auxiliary storage part 240 functions as a storage part, and has, for example, an HDD (Hard Disk Drive), an SSD (Solid State Drive), a flash memory, and the like. In this embodiment, personal information such as a name, belonging department, position, specialized field, approval authority, extension number, and e-mail address, and the like are stored for each member of the organization in the auxiliary storage part 240. In this specification, the organization means a related enterprise group, a unitary enterprise, a department or division within an enterprise, and the like. Information such as development contents and planning contents is shared within the organization. Further, the members mean members of the organization, and the members include the leaders (superior) and general members (subordinate) of the organization. Further, the auxiliary storage part 240 functions as a schedule recording part and records a business schedule for each member of the above organization. Incidentally, the personal information and business schedule for each member may be configured to be stored in the auxiliary storage part of the database server 300.

The communication part 250 has, for example, a communication device such as a network interface card (NIC) and transmits data to and from the database server 300 and the client terminal 400 through the communication network 101.

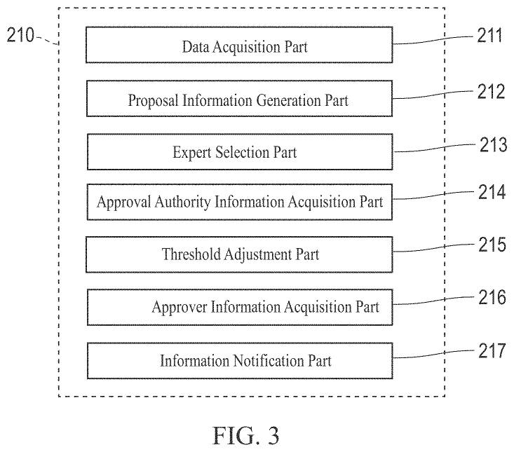

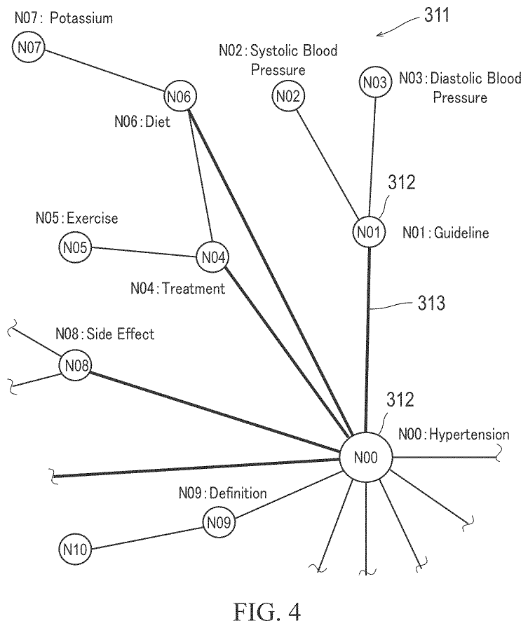

FIG. 3 is a schematic block diagram illustrating the main functional parts of the CPU 210 of the expert selection device 200 illustrated in FIG. 2. Further, FIG. 4 is a network diagram illustrating a part of the knowledge information stored in a knowledge database 310 illustrated in FIG. 1. The knowledge information is, for example, information obtained by making the relationship between the elements corresponding to a plurality of pieces of information forming the knowledge of one member into a knowledge network. The element can be, for example, a concept represented by a word, sentence, or clause. In this embodiment, the knowledge information is modeled as a knowledge network (or semantic network) in which the knowledge of one member is connected according to the relationship between a plurality of concepts.

As illustrated in FIG. 3, in this embodiment, the CPU 210 (hardware processor) executes the expert selection program to function as a data acquisition part 211, a proposal information generation part 212, an expert selection part 213, and an approval authority information acquisition part 214, a threshold adjustment part 215, an approver information acquisition part 216, and an information notification part 217. The outline of each function is as follows.

The data (hereinafter referred to as “proposal data”) of the proposal document created by the proposer is transmitted to the expert selection device 200. The data acquisition part 211 of the expert selection device 200 acquires the proposal data. The proposer means one or more members of the organization. The proposal data means unapproved data which needs to be approved by the approver. The approver means a member, who performs the approval procedure of the proposal data, other than the proposer of the above organization, and the direct supervisor of the proposer is selected in advance before the expert is selected.

The proposal information generation part 212 generates proposal information in which the relationships between the elements included in the proposal data acquired by the data acquisition part 211 are structured (for example, networked). Specific examples of the proposal information will be described later (see FIG. 8). The data acquisition part 211 and the proposal information generation part 212 function as a proposal information acquisition part.

The expert selection part 213 compares the proposal information generated by the proposal information generation part 212 and the knowledge information in which the relationships between the elements corresponding to the plurality of information forming the knowledge are structured (for example, networked) for each member other the proposer. Specific an example of the knowledge information of the member will be described later (see FIG. 4). Incidentally, the proposer and the member of a comparison target belong to the same organization.

The expert selection part 213 compares the proposal information and the knowledge information on the basis of a predetermined standard. The predetermined standard is, for example, that the proposal information and the knowledge information match or are similar. The similarity between the proposal information and the knowledge information is calculated, and whether or not the proposal information and the knowledge information are similar can be determined, for example, on the basis of whether or not the similarity is equal to or higher than a predetermined threshold value.

The expert selection part 213 selects an expert who has knowledge regarding the proposal data from a plurality of members on the basis of the comparison result. The selected experts may be one or more. In this embodiment, the selected expert can become an approver through the authorization of the supervisor. Alternatively, a configuration may be made in which the selected expert becomes the approver without the authorization of the supervisor. Further, the selected expert can be a supporter who supports the supervisor who is the approver with respect to the approval procedure of the proposal data. In this case, the expert can give advice to the supervisor, for example, about the contents of the proposal data and the propriety of approval of the proposal data.

The approval authority information acquisition part 214 reads and acquires the information regarding the approval authority stored in the auxiliary storage part 240. For example, the type or the like of document in which the approval authority is given to each member of the organization is defined in the information regarding the approval authority.

The threshold adjustment part 215 adjusts a predetermined threshold value. For example, in a case where it is determined by the approver that it is necessary to obtain immediate approval for the proposal data, the threshold adjustment part 215 performs an adjustment of reducing predetermined threshold value. For example, the case in which the immediate approval is required is a case where the interval between the date and time of the deadline of the approval procedure and the current date and time is smaller than the predetermined number of days. The predetermined number of days may vary depending on the type and contents of the proposal document, but may be, for example, about one day to one week. Accordingly, it is expected that the hurdle for selecting experts is lowered, and the number of selected experts increases. As a result, it is possible to avoid or reduce the situation in which all the selected experts cannot accept the approver due to, for example, the convenience of the business schedule or unforeseen circumstances.

In a case where the proposal document is presented to the outside of the organization (for example, one enterprise), the threshold adjustment part 215 performs an adjustment of raising a predetermined threshold value. This is because the presentation of the document to the outside of the organization requires the careful determination of an expert who is familiar with the contents of the document. When the predetermined threshold value is raised, members who have deep knowledge about the contents of the proposal document can be selected among the experts. Therefore, for example, in a case where the proposal document contains contents that is inappropriate for presentation to the outside of the organization, it is possible for the selected expert to find the inappropriate contents and urge the proposer to correct or delete the contents.

As described later, the contents of the proposal document are analyzed by the proposal information generation part 212. The threshold adjustment part 215 can determine whether or not the proposal document is presented to the outside of the organization on the basis of the analysis result of the proposal information generation part 212. Whether or not the proposal document is presented to the outside of the organization can be determined from, for example, the description regarding the submission destination of the proposal document or the contents (for example, joint development with other companies or the like) of the proposal document.

The approver information acquisition part 216 acquires information regarding the member set as an approver on a workflow system for the proposal data. The workflow system is a system which performs a process of approval procedure, settlement, or the like on electronic documents such as proposal documents according to a predetermined work procedure (workflow). On the workflow system, for example, a supervisor and a superior one level higher can be set as the approver. In the approval workflow on the workflow system, it is preferable that the approver includes an expert.

The information notification part 217 notifies information regarding the experts selected by the expert selection part 213. The notification destination can be the supervisor and/or the proposer. In particular, in a case where the selected expert is not the supervisor (that is, a member who has an approval authority for the proposal data), the information notification part 217 notifies the supervisor of the information regarding the expert. The name, personal number (an employee number in the case of enterprise), nickname of the expert and the like may be included in the information regarding the experts, and a belonging department, a position, an extension number, an e-mail address, and the like may be included in additional information. The supervisor confirms whether the selected expert is suitable as the approver or supporter of the proposal document by referring to the information regarding the expert. In a case where it is required to authorize the approver, if the selected expert is suitable as the approver or supporter, the supervisor authorizes the expert as the approver, or if not, the supervisor does not authorize the expert. Further, in a case where the selected expert is authorized as the approver, the supervisor requests the approval procedure of the proposal document to the expert (grants the approval authority to the requested expert).

<Database Server 300>

The database server 300 is a server (computer) having the knowledge database 310. Since the database server 300 has the same hardware configuration as that of the expert selection device 200, detailed description thereof will be omitted. The knowledge database 310 is stored in the auxiliary storage part.

In the knowledge database 310, the knowledge information of a plurality of members is stored for each individual. The knowledge information is generated or updated on the basis of the work information based on the usual work of the user on the client terminal 400 or the usual work of another user on another client terminal and is stored in the knowledge database 310.

The knowledge information includes a plurality of elements and a relationship (connection) between the elements. For example, as illustrated in the network diagram of FIG. 4, in the knowledge information 311 which is a part of the knowledge information of a certain member, each element can be represented by a node (indicated by a circle) 312, and the connection between the nodes 312 can be represented by a line 313 connecting the nodes 312. For example, nodes N00, N01, N04, N08, and N09 correspond to the elements “hypertension”, “guideline”, “treatment”, “side effect”, and “definition”, and the nodes N01, N04, N08, and N09 are connected with node N00. The relationship between nodes includes the relationship between upper/lower concepts (for example, a connection source node is the upper concept, and a connection destination node is the lower concept), the relationship in which the connection destination node is an essential attribute or an arbitrary attribute of the connection source node in a case where the connection destination node is an attribute of the connection source node, a case where the connection destination node is an arbitrary possible value of the connection source node, and the like.

The knowledge information may include information indicating the relative importance of each element in a group (hereinafter, also referred to as “element group”) of elements associated with each other. An element of which the importance is higher than that of other elements can be, for example, a starting point or a main point of knowledge represented by the knowledge information. For example, an element which has many connections to other elements is considered to be relatively important. Therefore, the importance of an element can be determined by the number of connections with other elements. Further, the knowledge database 310 may be configured to store the importance of the elements numerically.

On the network diagram, for example, a node having many connections to other nodes is drawn larger than a node having few connections. In the example illustrated in FIG. 4, the node N00 representing “hypertension” is drawn larger than the other nodes since the node N00 is connected to the nodes N01, N04, N08, and N09 and has many connections to other nodes. Further, in the example of FIG. 4, the node N00 is the starting point of the knowledge represented by the knowledge information 311.

The knowledge information may also include information indicating the strength of the connection between the elements. The strength of the connection between the elements can also be stored numerically in the knowledge database 310. On the network diagram, for example, when the connection is strengthened, a line is drawn thick.

In response to the request from the expert selection device 200, the database server 300 reads the knowledge information of the member from the auxiliary storage part and transmits the information to the client terminal 400.

Incidentally, in the above example, the knowledge network model of the element group is illustrated. However, the knowledge information may be modeled as a network (semantic network) in which the relationship (inclusion relationship or the like) of connection is considered as well as the presence/absence of connection between the elements and the strength of connection.

<Client Terminal 400>

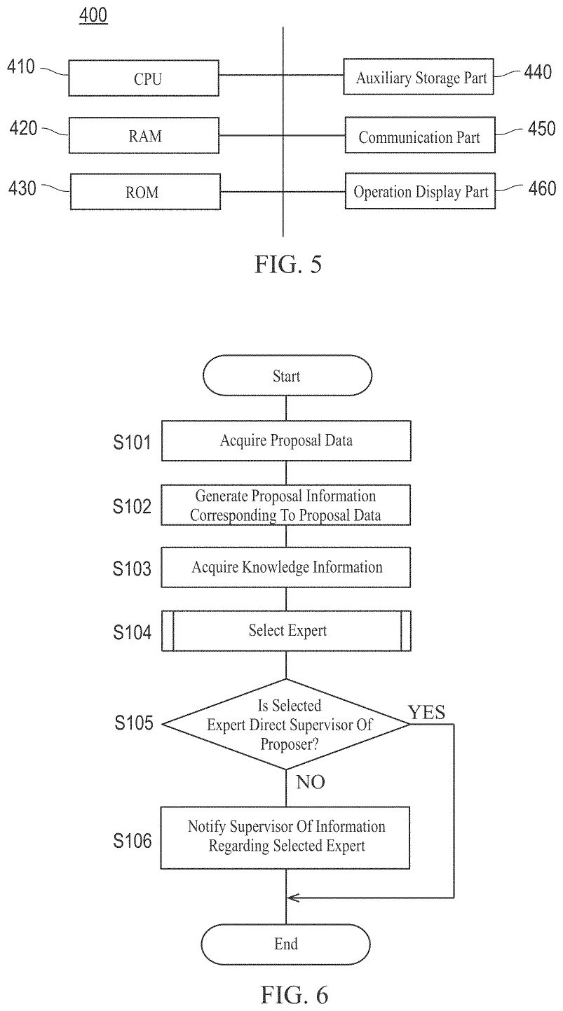

FIG. 5 is a schematic block diagram illustrating the hardware configuration of the client terminal 400 illustrated in FIG. 1.

As illustrated in FIG. 5, the client terminal 400 is, for example, a computer including a CPU 410, a RAM 420, a ROM 430, an auxiliary storage part 440, a communication part 450, an operation display part 460, and the like. The client terminal 400 may be, for example, a personal computer, a PDA (Personal Digital Assistant), a smartphone, or the like.

The hardware configurations of the CPU 410, the RAM 420, the ROM 430, the auxiliary storage part 440, and the communication part 450 are the same as the hardware configurations of the CPU 210, the RAM 220, the ROM 230, the auxiliary storage part 240, and the communication part 250 of the expert selection device 200, respectively. Thus, the detailed description thereof will be omitted.

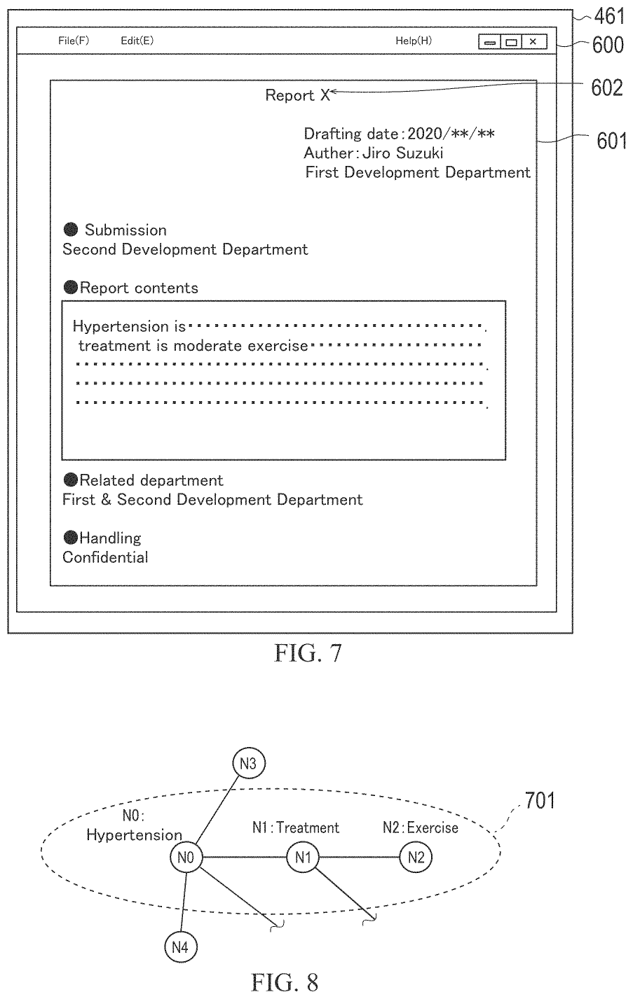

The operation display part 460 has an input part and an output part. The input part includes, for example, a keyboard, a mouse, and the like, and is used for the user to perform various instructions (inputs) such as character input by the keyboard, the mouse, and the like, and various settings. Further, the output part includes a display 461 (see FIG. 7) and is used to present a document or the like created by application software (hereinafter, simply referred to as “application”) to the user. Further, in this embodiment, in response to the instruction of the CPU 410, the output part displays information regarding the expert on the display 461 to present the information to the user. Further, the output part has a speaker and can provide the user with the information regarding the expert by voice.

<Control Method of Expert Selection System>

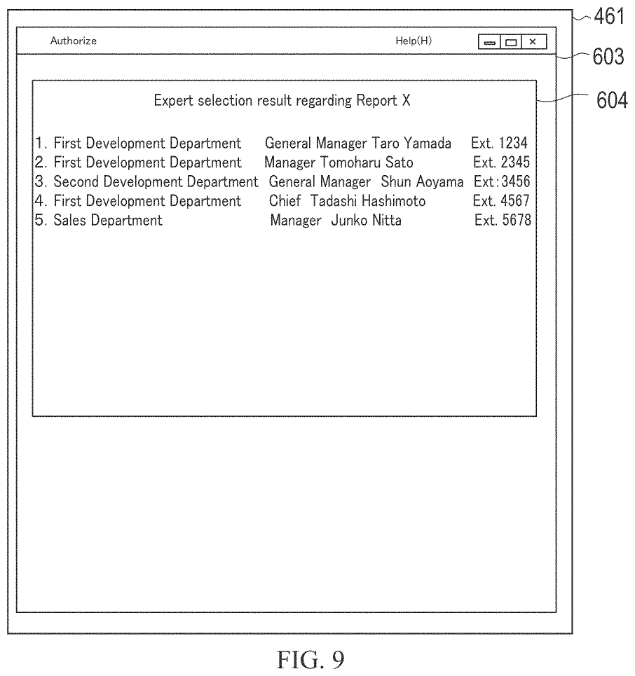

FIG. 6 is a flowchart illustrating a schematic processing procedure of the control method of the expert selection system 100 according to one embodiment. The processing of the flowchart of FIG. 6 is realized by the CPU 210 executing the expert selection program. FIG. 7 is a schematic diagram illustrating a report created by the proposer, and FIG. 8 is a network diagram illustrating the proposal information corresponding to the report of FIG. 7. Further, FIG. 9 is a schematic diagram illustrating the display of the information regarding the expert.

As illustrated in FIG. 6, first, the proposal data is acquired (step S101). The proposer is, for example, a user of the client terminal 400 and creates a proposal document by using an application running on the client terminal 400. For example, software such as document creation, drawing, programming, and CAD (Computer-Aided Design) may be included in the applications, and files in various formats created by these applications may be included in the proposal document.

For example, it is assumed that the proposer creates a report as a proposal document and applies for approval. As illustrated in FIG. 7, as an example, the proposer edits a report 601 by using document creation software on the client terminal 400. The report 601 being edited on an editing screen 600 of the document creation software is displayed on the display 461. “Report X” 602 as the title of the document and “submission destination”, “report contents”, “related department”, and “handling” as items are included in the report 601. Further, “hypertension is . . . treatment is moderate exercise . . . ” input by the proposer is displayed in the item of “report contents”.

After the editing of report 601 is completed, the proposer performs the operation of approval application from the operation menu of the workflow system in order to obtain approval from the approver who has the approval authority for this report 601 (proposal data). The data (proposal data) of the report 601 is transmitted to the expert selection device 200 through the communication part 450. The data acquisition part 211 acquires the proposal data from the client terminal 400 through the communication part 250.

Next, the proposal information corresponding to the proposal data is generated (step S102). The proposal information generation part 212 comprehensively analyzes the proposal data and performs processing according to the proposal data to generate proposal information. For example, the proposal information generation part 212 performs natural language processing by using keywords such as words included in the proposal data as elements, analyzes the processing result, and generates proposal information in which the elements are structured by using a meaning vector or the like. For example, the proposal information may be a proposal information network in which the relationships between the elements included in the proposal data are networked.

Natural language processing is a technique of recognizing and generating a natural language used by humans by a computer, and for example, an existing technique such as BERT (Bidirectional Encoder Representations from Transformers) can be used. In the example illustrated in FIG. 7, the proposal information generation part 212 performs the natural language processing on the report 601 to extract elements and the relationships between the elements by using the keywords included in the report 601 as the elements and generates proposal information.

The proposal information generation part 212 vectorizes the report 601 at the word level or the character level and estimates the potential meaning of the report 601 from the combination of each word by a topic model to recognize the field of the proposal document.

As illustrated in FIG. 8, since the item of “report contents” of the report 601 is described, the proposal information generation part 212 extracts, for example, “hypertension”, “treatment”, and “exercise” as elements and networks the relationship therebetween to generate proposal information 701. In the same drawing, each element of “hypertension”, “treatment”, and “exercise” is represented by the nodes N0, N1, N2, respectively. Further, the nodes N0 and N1 are connected with a line, and the nodes N1 and N2 are connected with a line, whereby the relationships between the connected nodes are represented. In the example illustrated in FIG. 8, “treatment” is associated as a matter related to “hypertension”, and “exercise” is associated as a matter related to “treatment”.

The contents of the source code of the program created by the user, the language of the source code, an architecture, and the like can also be a target to be analyzed. Further, the data created by CAD, illustration creation software, design software, and the like can also be a target.

Returning to FIG. 6 again, the knowledge information is acquired (step S103). The expert selection part 213 acquires the proposal information 701 from the proposal information generation part 212 and acquires the knowledge information of the member who is a target to be compared with the proposal information 701 from the database server 300. Herein, the member as a comparison target may be all members in the organization or may be some members in the organization such as members belonging to a specific department.

Incidentally, the expert selection part 213 may read the knowledge information of a plurality of members as the comparison target one by one from the database server 300, and may read the knowledge information of a predetermined number of members at a time in a case where the number of members as the comparison target is large. The read knowledge information is stored in the RAM 220.

Next, an expert is selected (step S104). The expert selection part 213 compares the proposal information 701 with the knowledge information of the members of the comparison target compared stored in the RAM 220 and determines an expert from the members of the comparison target on the basis of the comparison result. Details of a method of determining an expert will be described later on the basis of the comparison result.

Next, it is determined whether or not the selected expert is the direct supervisor of the proposer (step S105). The information notification part 217 determines whether or not the expert selected by the expert selection part 213 is the direct supervisor of the proposer. In a case where the selected expert is the supervisor (step S105: YES), the process ends (end).

On the other hand, in a case where the selected expert is not the supervisor (step S105: NO), the information notification part 217 notifies the supervisor of the information regarding the selected expert (step S106). The information notification part 217 transmits the information (for example, a belonging department, a name, a specialized field, a contact address, and the like) regarding the expert to the client terminal 400 of the supervisor. In the client terminal 400 of the supervisor, the CPU 410 controls the operation display part 460 so that the information regarding the expert is displayed on the display 461.

For example, as illustrated in FIG. 9, the information 604 regarding the expert is displayed on a screen 603 of the approval workflow of the workflow system.

The supervisor confirms the information regarding the expert displayed on the display 461. Further, in a case where it is required to authorize the selected expert, the supervisor determines whether or not the selected expert is suitable as the approver of the proposal document. In a case where it is determined to be suitable, the supervisor authorizes the selected expert, or in a case where it is not determined to be suitable, the supervisor does not authorize the selected expert. Further, in a case where the selected expert is authorized, the supervisor requests the approval procedure of the proposal document to the selected expert.

Incidentally, in a case where the selected experts are plural, the supervisor may or may not authorize each of the selected experts. Accordingly, the supervisor substantially can select one or more final approvers from among the selected experts.

In this way, according to the processing of the flowchart illustrated in FIG. 6, proposal data is acquired, the proposal information of the acquired proposal data is generated, the generated proposal information and the knowledge information are compared for each member other than the proposer, and an expert is selected from a plurality of members of the organization on the basis of the comparison result.

<Processing to Select Experts>

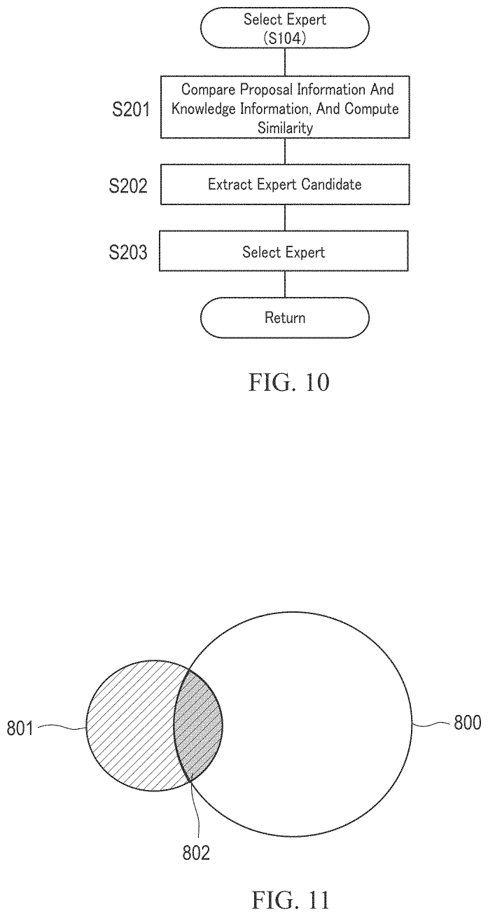

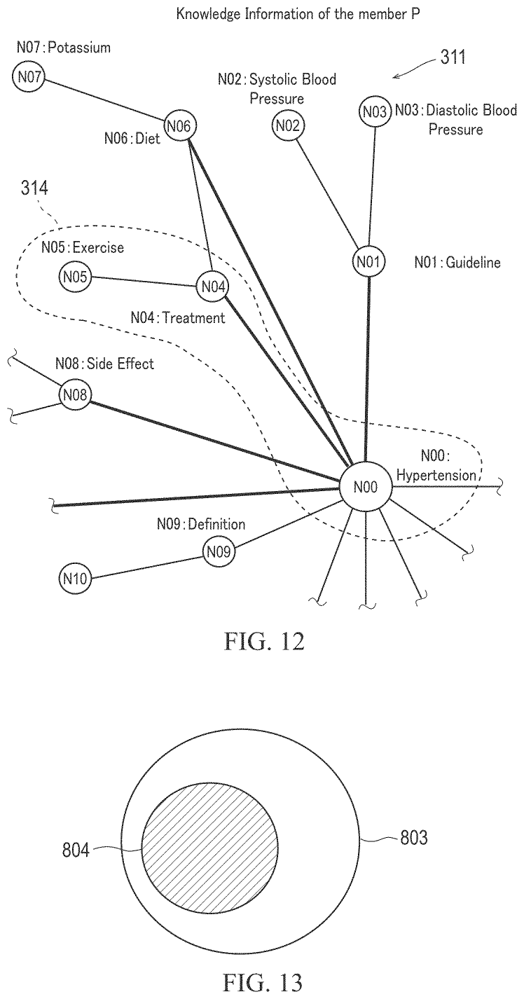

FIG. 10 is a subroutine flowchart illustrating the details of the step (S103) of selecting an expert in FIG. 6. The processing of the subroutine flowchart illustrated in FIG. 10 is realized by the CPU 210 executing the expert selection program. FIG. 11 is a conceptual diagram illustrating the concept of a similarity between the proposal information and the knowledge information, and FIG. 12 is a network diagram illustrating the comparison between the proposal information and the knowledge information. Further, FIG. 13 is a conceptual diagram illustrating an inclusion relationship between the proposal information and the knowledge information.

As illustrated in FIG. 10, the proposal information and the knowledge information are compared, and a similarity is calculated (S201). The similarity is an index indicating the degree to which the proposal information and the knowledge information are similar. As illustrated in FIG. 11, for example, in a region 800 of the concept represented by the knowledge information of a member and a region 801 of the concept represented by the proposal information, the similarity can be represented by a distance between the centers of the two regions, or a region 802 where the two regions overlap.

That is, when the distance between the centers of the two regions decreases, the similarity increases, and when the distance increases, the similarity decreases. Further, the overlapping region 802 of the two regions is enlarged, the similarity increases, and when the region 802 is reduced, the similarity decreases. The region 802 is a region defined by the element groups contained in this region and the relationships between them. Incidentally, the region 800, the region 801 and the region 802 are usually multidimensional regions but are represented as two-dimensional regions for the sake of simplicity in FIG. 11.

It is also possible to calculate the similarity on the basis of the vector of the elements included in the proposal information and the vector of the elements included in the knowledge information. In this specification, both vectors are referred to as a proposal information vector and a knowledge information vector, respectively. The vector of the proposal information may be the average value or the representative value of the vectors of the plurality of elements included in the proposal information, and the vector of the knowledge information may be the average value or the representative value of the vectors of the plurality of elements included in the knowledge information.

More specifically, for the proposal information, the expert selection part 213 converts a plurality of elements included in the proposal information into vector values by using a method such as Word2Vec, for example, calculates the average value of those vectors, and defines the value as the vector of the proposal information. Further, for the knowledge information, as in the case of the proposal information, a plurality of elements included in the knowledge information are converted into vector values, the average value of those vectors is calculated, and the value is defined as the vector of the knowledge information. Then, the expert selection part 213 obtains a cosine similarity on the basis of the vector of the proposal information and the vector of the knowledge information. The cosine similarity between the vector of the proposal information and the vector of the knowledge information is also called a distance between the two vectors.

Next, an expert candidate is extracted (step S202). In a case where it is determined as a result of comparing the proposal information 701 and the knowledge information for each member of the comparison target that both pieces of information match or are similar, the expert selection part 213 extracts the member of the knowledge information as an expert candidate.

For example, as illustrated in FIG. 12, it assumed that a member P has experience in research and development on the treatment of hypertension, and each element of “hypertension”, “treatment”, and “exercise” is included in the knowledge information 311 which is a part of the knowledge information of the member P. On the basis of FIGS. 8 and 12, when comparing the proposal information 701 and the knowledge information 311 of the member P, the nodes N0, N1 and N2 of the proposal information 701 correspond to the nodes N00, N04 and N05 of the knowledge information 311, respectively. Further, the line connecting the node NO and the node N1 of the proposal information 701 corresponds to the line connecting the node N00 and the node N04 of the knowledge information 311, and the line connecting the node N1 and the node N2 of the proposal information 701 corresponds to the line connecting node N04 and node N05 of the knowledge information 311. Therefore, the proposal information 701 corresponds to a knowledge network 314 having the node N00 of the knowledge information 311 of the member P as a starting point.

That is, as illustrated in FIG. 13, when the concept represented by the knowledge information 311 of the member P is represented by the region 803, and the concept represented by the proposal information 701 is represented by the region 804, the region 804 is included in the region 803. That is, the member P has the concept (knowledge) represented by the proposal information 701, and the proposal information is determined to be similar to the knowledge information.

As illustrated in FIG. 11, in a case where the region 801 overlaps with the region 800 in a part of the region 802, and the distance between the center of the region 800 and the center of the region 801 is less than or equal to a predetermined distance, the proposal information is determined to be similar to the knowledge information. Alternatively, in a case where the region 802 is greater than or equal to a predetermined area, the proposal information is determined to be similar to the knowledge information, and the region 802 is less than a predetermined area, the proposal information is determined not to be similar to the knowledge information.

In a case where a similarity is calculated on the basis of the vector of the proposal information and the vector of the knowledge information, it is determined that the proposal information and the knowledge information are similar when the calculated similarity is larger than a predetermined threshold value, and it is determined that the proposal information and the knowledge information are not similar when the similarity is smaller than a predetermined threshold value.

Next, an expert is selected (step S203). For example, the expert selection part 213 can select a member who satisfies a predetermined condition as an expert from the expert candidates extracted in step S202. The predetermined condition may be, for example, having an approval authority of the proposal document. On the basis of the information regarding the approval authority stored in the auxiliary storage part 240, the expert selection part 213 selects a member who has the approval authority for the proposal document as an expert from among the expert candidates. Further, the expert may be selected from members who have the approval authority for the proposal document and belong to the same department, members (for example, working on another client terminal 400) who are currently present, and the like among the expert candidates.

The expert selection part 213 can select a member who is closest to the proposer in the hierarchy of the organization to which the proposer belongs and is in a higher position than the proposer as the expert from the extracted expert candidates. In this case, having approval authority may be set as the condition or may not be set as the condition. Further, being close to the proposer in the hierarchy of the organization means, for example, belonging to the same department or the same team.

The predetermined condition may be the time required to scrutinize the proposal document. The expert selection part 213 can read a business schedule for each member from the auxiliary storage part 240 and preferentially select a member who have a vacancy in the work schedule and can secure the time required to scrutinize the proposal document as the expert from the extracted expert candidates. For example, in a case where there is a vacancy of about half a day to several days in the business schedule depending on the type and contents of the proposal document between the date and time of the deadline for the approval procedure and the current date and time, it is determined that there is a vacancy in the business schedule. Incidentally, the schedule of business members may be acquired from a commonly used scheduler.

The expert selection part 213 can also select a member who is extracted as the expert candidate in step S202 and set as the approver on the workflow system as a final expert. As a result, the members set as approvers on the workflow system used conventionally can be narrowed down to the members who have knowledge regarding the contents of the proposal document. Therefore, it is possible to prevent a member who is set as the approver on the workflow system but does not have sufficient knowledge regarding the contents of the proposal document from becoming an approver.

A configuration may be made such that the proposer selects the final (formal) expert from the extracted expert candidates.

As described above, in the processing of the subroutine flowchart illustrated in FIG. 10, the proposal information and the knowledge information are compared, and the similarity is calculated. Then, in a case where the proposal information and the knowledge information match or are similar, the members of the knowledge information are extracted as the expert candidates. Further, an expert is selected from the extracted expert candidates.

Incidentally, in the above example, a case is described in which in a case where the proposal information and the knowledge information match or are similar, the expert selection part 213 extracts the members of the knowledge information as the expert candidates and selects the expert from the expert candidates. However, this embodiment is not limited to such a case. In a case where it is determined as a result of comparing the proposal information and the knowledge information that both pieces of information match or are similar, the expert selection part 213 can select the member of the knowledge information as the expert. That is, all members having the knowledge information which matches or is similar to the proposal information can be selected as the expert.

According to the expert selection device 200 of this embodiment described above, the expert on the proposal document is selected on the basis of the result of comparing the proposal information based on the proposal document and the knowledge information stored for each member. Therefore, the selected expert can act as the approver and perform the approval procedure of the proposal document. As a result, it is possible to prevent a situation in which the approver is unable to fully fulfill the role of the approver due to the lack of knowledge regarding the proposal document.

As described above, the expert selection system 100 and the expert selection program have been described in the embodiment. However, the present invention can be appropriately added, modified, and omitted by those skilled in the art within the scope of the technical idea.

For example, in the above-described embodiment, a case has been described in which the knowledge information of the members is read from the database server 300 to the expert selection device 200, and the proposal information and the knowledge information are compared in the expert selection part 213 of the expert selection device 200. However, the invention is not limited to such a case, and a configuration may be made in which the proposal information and the knowledge information are compared in the database server 300.

In the above-described embodiment, a case has been described in which the database server 300 has the knowledge database 310. However, a configuration may be made in which the expert selection device 200 has the knowledge database.

The expert selection program may be provided by a computer-readable recording medium such as a USB memory, a flexible disk, and a CD-ROM or may be provided online via a network such as the Internet. In this case, the program recorded on the computer-readable recording medium is usually transferred to a memory, a storage, or the like and stored. Further, this expert selection program may be provided, for example, as independent application software or may be incorporated into the software of each device as one function of the server.

Further, some or all of the processing executed by the expert selection program in the embodiment may be replaced with hardware such as a circuit to be executed.

Although embodiments of the present invention have been described and illustrated in detail, the disclosed embodiments are made for purposes of illustration and example only and not limitation. The scope of the present invention should be interpreted by terns of the appended claims.

1. An expert selection system comprising:
a storage part that stores knowledge information in which relationships between elements corresponding to a plurality of pieces of information forming knowledge are structured for each member; and
a hardware processor, wherein
the hardware processor acquires proposal information in which relationships between elements included in proposal data of a proposer are structured, compares the proposal information and the knowledge information associated with members other than the proposer of the proposal data for each member, and selects an expert who has knowledge regarding the proposal data from a plurality of the members on the basis of a comparison result.

2. The expert selection system according to claim 1, wherein
the proposal data is unapproved data which needs to be approved by an approver, and
the expert is the approver who performs an approval procedure of the unapproved data or a supporter who supports the approver who performs the approval procedure of the unapproved data with respect to the approval procedure of the unapproved data.

3. The expert selection system according to claim 1, wherein
the hardware processor
acquires information regarding an approval authority of the plurality of members in an organization to which the proposer and the plurality of members belong,
extracts, in a case where it is determined as a result of comparing the proposal information and the knowledge information that both pieces of information match or are similar, members of the knowledge information as expert candidates, and
selects a member having an approval authority for the proposal data as the expert from the extracted expert candidates on the basis of the acquired information regarding the approval authority.

4. The expert selection system according to claim 1, wherein
the hardware processor
extracts, in a case where it is determined as a result of comparing the proposal information and the knowledge information that both pieces of information match or are similar, members of the knowledge information as expert candidates, and
selects a member who is closest to the proposer in a hierarchy of an organization to which the proposer and the plurality of members belong and is in a higher position than the proposer as the expert from the extracted expert candidates.

5. The expert selection system according to claim 1, wherein
the hardware processor notifies information regarding the expert.

6. The expert selection system according to claim 5, wherein
the proposal data is unapproved data which needs to be approved by an approver,
the expert is the approver who performs an approval procedure of the unapproved data or a supporter who supports the approver who performs the approval procedure of the unapproved data with respect to the approval procedure of the unapproved data, and
the hardware processor
notifies the supervisor of the information regarding the expert in a case where the selected expert is not a direct supervisor of the proposer.

7. The expert selection system according to claim 2, wherein
the hardware processor
determines that the proposal information and the knowledge information are similar in a case where it is determined whether or not a similarity between a vector of elements included in the proposal information and a vector of elements included in the knowledge information is equal to or higher than a predetermined threshold value, and it is determined that the similarity is equal to or higher than the predetermined threshold value.

8. The expert selection system according to claim 7, wherein
the hardware processor adjusts the predetermined threshold value and, in a case where it is determined that it is necessary to obtain immediate approval for the proposal data, performs an adjustment of reducing the predetermined threshold value.

9. The expert selection system according to claim 7, wherein
the hardware processor adjusts the predetermined threshold value and, in a case where the proposal data is presented to an outside of an organization to which the proposer and the plurality of members belong, performs an adjustment of raising the predetermined threshold value.

10. The expert selection system according to claim 3, further comprising:
a schedule recording part that records business schedules of the plurality of members, wherein
the hardware processor
preferentially selects a member who has a vacancy in a schedule as the expert from the extracted expert candidates.

11. The expert selection system according to claim 3, wherein
the hardware processor acquires information regarding a member set as an approver on a workflow system with respect to the proposal data and selects a member who is extracted as the expert candidate and is set as the approver on the workflow system as the expert.

12. The expert selection system according to claim 1, wherein
the hardware processor
selects a member of the knowledge information as the expert in a case where it is determined as a result of comparing the proposal information and the knowledge information that both pieces of information match or are similar.

13. The expert selection system according to claim 1, wherein
the proposal information is information in which the relationships between the elements included in the proposal data are networked.

14. The expert selection system according to claim 1, wherein
the knowledge information is information in which the relationships between the elements corresponding to the plurality of pieces of information forming the knowledge are networked.

15. A non-transitory recording medium storing a computer readable program causing a computer to perform:
acquiring proposal information in which relationships between elements included in proposal data of a proposer are structured;
acquiring knowledge information in which relationships between elements corresponding to a plurality of pieces of information forming knowledge are structured; and
comparing the proposal information of the proposer and the knowledge information associated with members other than the proposer for each of the members and selecting an expert who has knowledge regarding the proposal data from a plurality of the members on the basis of a comparison result.

16. The non-transitory recording medium storing a computer readable program according to claim 15, wherein
the proposal data is unapproved data which needs to be approved by an approver, and
the expert is the approver who performs an approval procedure of the unapproved data or a supporter who supports the approver who performs the approval procedure of the unapproved data with respect to the approval procedure of the unapproved data.

17. The non-transitory recording medium storing a computer readable program according to claim 15, the program causing a computer to further perform:
notifying the supervisor of information regarding the expert in a case where the selected expert in the comparing is not a direct supervisor of the proposer of the proposal data.
